(19) United States
(12) Patent Application Publication
(10) Pub. No.: US 2023/0025360 A1
(43) Pub. Date: Jan. 26, 2023

(54) PROCESSES FOR GENERATING SUPERIOR ANTI-TUMOR T-CELL EFFECTOR AND MEMORY CELLS

(71) Applicant: MUSC Foundation for Research Development, Charleston, SC (US)

(72) Inventors: Shikhar Mehrotra, Mount Pleasant, SC (US); Pravin Kesarwani, Royal Oak, MI (US); Shilpak Chatterjee, Charleston, SC (US)

(21) Appl. No.: 17/817,125

(22) Filed: Aug. 3, 2022

Related U.S. Application Data
(63) Continuation of application No. 16/095,791, filed on Oct. 23, 2018, now abandoned.
(60) Provisional application No. 62/328,780, filed on Apr. 28, 2016.

(51) Int. Cl. A61K 35/17; A61P 35/00; A61K 31/381; A61K 31/401; A61K 38/06; A61K 38/20; A61K 38/43; C12N 5/0783

(52) U.S. Cl. CPC A61K 35/17; A61P 35/00; A61K 31/381; A61K 31/401; A61K 38/063; A61K 38/2026; A61K 38/43; C12N 5/0636; C12N 2510/00

(57) ABSTRACT

The present invention relates to an adoptive T cell therapy using cells generated by genetically or biochemically reprogramming T cells to produce T memory stem cells (Tscm) that persist longer in vivo and are superior anti-tumor effector cells.

### CROSS REFERENCE TO RELATED APPLICATION

This application is a divisional of U.S. patent application Ser. No. 16/095,791, filed Oct. 23, 2018, which is the U.S. national phase application filed under 35 U.S.C. § 370 claiming benefit to International Patent Application No. PCT/US17/30062, filed Apr. 28, 2017, which is in turn entitled to priority under 35 U.S.C § 119(e) to U.S. provisional application No. 62/328,780, filed Apr. 28, 2016, each of which is hereby incorporated by reference in its entirety.

### STATEMENT REGARDING FEDERALLY SPONSORED RESEARCH OR DEVELOPMENT

This invention was made with government support under AG09215, AG017586, and NS053488 awarded by the National Institute of Health. The government has certain rights in the invention.

### BACKGROUND OF THE INVENTION

Adoptive T cell therapy (ACT) is a promising approach for treating patients with advanced malignancies (Rosenberg, S. A. 2012, Sci Transl Med, 4(127):127p58; Kershaw, M. H. et al., 2013, Nat Rev Cancer, 13(8):525-41). However, ACT is only able to cure a small proportion of the patients treated (Rosenberg, S. A. 2012, Sci Transl Med, 4(127):127ps8; Phan, G. Q. et al., 2013, Cancer Control, 20(4):289-97; Gajewski, T. F. et al., 2013, Curr Opin Immunol, 25(2):268-76; Gajewski, T. F. et al., 2013, Nat Immunol, 14(10):1014-22), leaving substantial room for improvement. A rapid expansion step is usually employed to obtain a large number of T cells (1-1,000×10⁸ cells for ACT). The majority of T cells subjected to rapid expansion have an effector memory phenotype (Tem) with increased susceptibility to undergo activation induced cell death (AICD) (Mehrotra, S. et al. 2009, Adv Cancer Res, 102:197-227). Therefore, the recent strategies have focused on altering the T cell expansion protocols to generate central memory (Tcm) phenotype cells that persist longer and exhibit better tumor control (Kesarwani, P. et al., 2014, Cancer Res, 74(21):6036-47). Among the approaches used to program the expanding T cells towards a Tcm phenotype is to block the mTOR (Araki, K. et al., 2009, Nature, 460(7251):108-12), the Akt (van der Waart, A. B. et al., 2014, Blood, 124(23):3490-500), or the glycolytic pathways (Sukumar, M. et al., 2013, J Clin Invest, 123(10):4479-88). Another approach designed to increase the therapeutic efficacy of T cells for ACT is to reprogram the expanding T cells towards a ‘memory stem cells’ (Tscm) phenotype (Gattinoni, L. et al., 2012, Nat Rev Cancer, 12(10):671-84). Tscm cells are reported to have increased persistence and far improved tumor control due to their pluripotent or “stem-cell like” properties (Gattinoni, L. et al., 2012, Nat Rev Cancer, 12(10):671-84). Therefore, any T cell expansion process that programs the T cells towards a Tcm or a Tscm phenotype has a real potential to improve the effectiveness of ACT.

Thus, there is an urgent need in the art for compositions and methods for generating improved therapeutic cells for adoptive T cell therapy. The present invention addresses this need.

### SUMMARY OF THE INVENTION

In one aspect, the invention provides a method of generating T memory stem cells (Tscm) that persist long term in vivo and exhibit superior anti-cancer activity, the method comprising reprogramming T cells to exhibit higher expression of cell surface thiols. In one embodiment, reprogramming T cells comprises genetic modification of the T cells to express thioredoxin. In another embodiment, reprogramming T cells comprises contacting the T cells with a pharmacological modulator selected from the group consisting of IL-4, recombinant thioredoxin (rTrx), thioredoxin-reductase, glutathione, α-ketoglutarate, and proline. In various embodiments, the Tscm cells persist in vivo for at least four months, five months, six months, seven months, eight months, nine months, ten months, eleven months, twelve months, two years, or three years after administration. In one embodiment, the invention comprises a cell created by any of the methods disclosed herein.

In one aspect, the invention provides a method of treating cancer in a mammal, the method comprising administering an effective amount of a reprogrammed T cell to a mammal in need thereof, wherein the reprogrammed T cell exhibits a higher expression of cell surface thiols.

In one aspect, the invention provides a method for stimulating an immune response to a target cell population or tissue in a mammal, comprising administering to a mammal an effective amount of a reprogrammed T cell that exhibits higher expression of cell surface thiols, thereby stimulating a response to a target cell population or tissue in the mammal.

In one aspect, the invention provides a method of providing an anti-tumor immunity in a mammal, the method comprising administering to the mammal an effective amount of a reprogrammed T cell that exhibits higher expression of cell surface thiols, thereby providing an anti-tumor immunity in the mammal.

In one aspect, the invention provides a method of generating a memory immune response in a mammal, the method comprising co-administering a first population of antigen reactive T cells and a second population of antigen reactive T cells modified to exhibit higher expression of cell surface thiols.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts a schematic diagram. It highlights the differences between conventional T cells (FIG. 1A) and Trx overexpressing T cell (FIG. 1B), and how secreted Trx in vivo or using Trx during ex vivo programming may alter the anti-oxidant state and modulate signaling proteins due to increased expression of reduced thiols (—SH) (FIG. 1C), which in turn would correlate to improved tumor control.

FIG. 2 depicts results from example experiments, demonstrating Trx overexpressing transgenic T cells exhibit higher anti-oxidant capacity and reduced susceptibility to oxidative stress. (FIG. 2A). The gel picture shows PCR based genotyping for Pmel and Pmel-Trx mice. (FIG. 2B). Trx expression staining on the Pmel and Pmel-Trx T cells. N=5. (FIG. 2C). Upper panel shows increased expression of cell surface thiols (c-SH) with a concomitant decrease in intracellular reactive oxygen species accumulation (H₂O₂ by DCFDA) in Pmel-Trx cells as compared to Pmel T cells. Numerical values are MFI. Lower panel shows lower cell death in Pmel-Trx T cells in presence of 50 μM of exogenous H₂O₂ (left), or after restimulation with cognate antigen (right). Numerical values represent gated percent cells. N=5. (FIG. 2D). Melanoma epitope gp100 reactive T cells from Pmel and Pmel-Trx splenocytes were activated with cognate peptide for three days before being transferred i.p. to the EL4 ascitis established for 14 days in C57BL/6 mice. The T cells were retrieved after 24 hrs. and oxidative stress markers 8-OGdG (left panel) and Nitrotyrosine (right panel) were determined in V1312 gated CD8+ T cells. Numerical values on lower right corner are mean fluorescence intensity (MFI). N=2. *p value<0.05, **p value<0.01.

FIG. 3 depicts results from example experiments, demonstrating cell signaling and function of Pmel-Trx T cells. Pmel and Pmel-Trx derived splenic T cells were activated for three days with cognate antigen and used for determining: (FIG. 3A) Phosphorylation levels of key signaling molecules by intracellular staining with phospho-Abs (from BD) as per the manufacturers protocol. (FIG. 3B). Cells were left in IL2 (50 IU) for 15 minutes and 60 minutes before staining with fluorochrome conjugated antibody for phospho-STAT5 activation. (FIG. 3C) Intracellular IFNγ staining after reactivation with cognate antigen overnight. (FIG. 3D) Glucose uptake (using 2NBDG) after antigen restimulation for 4 hrs. (FIG. 3E) mRNA expression of various transcripts using the RNA isolated from the three day activated T cells. N=3. *p<0.05. (FIG. 3F) Pmel and Pmel-Trx T cells were activated with cognate antigen in presence of rIL-2 (50 units/ml) and used for determining oxygen consumption rate (OCR, left panel), spare respiratory capacity (SRC, middle panel), and extracellular acidification rate (ECAR) using real-time metabolic flux analyzer. Right panel shows ratio of OCR/ECAR.

FIG. 4 depicts results from example experiments, demonstrating metabolic profiling of Pmel-Trx T cells. (FIG. 4A) Ten million of three day activated Pmel and Pmel-Trx T cells were sorted and the pellets were snap frozen, and metabolite analysis was done using the Gas Chromatography-Mass Spectroscopy (GC-MS). The Principal Component Analysis (PCA) shows distribution of the metabolites. (FIG. 4B) Hierarchical distribution of the metabolites evaluated between the triplicate Pmel and Pmel-Trx T cells is shown in the heat map. (FIG. 4C) List of metabolites that were significantly (p value<0.05) upregulated in Pmel-Trx cells as compared to the Pmel T cells is shown. N=3.

FIG. 5 depicts results from example experiments, demonstrating increased glutamine dependence of Trx overexpressing T cells. The Pmel and Pmel-Trx T cells activated using cognate peptide antigen for three days were used to: (FIG. 5A) obtain RNA and determine the mRNA transcripts for amino acid transporters. The data presented are from three experiments. (FIG. 5B) Uptake of radiolabeled glutamine by Pmel and Pmel-Trx cells was measured as count per minute (CPM). Data are means f SD of four samples from one experiment and are representative of at least three independent experiments.

FIG. 6 depicts results from example experiments, demonstrating co-injecting Pmel-Trx and Pmel T cell controls tumor growth, and establishes anti-tumor T cell memory. (FIG. 6A) Schematic representation of Pmel (Thy1.1) and Pmel-Trx (Thy1.2) cells that were activated with cognate antigen for three days before mixing in 1:1 ratio, and adoptively transferred to the murine melanoma B16-F10 bearing immunocompetent C57BL/6 recipient mice. (FIG. 6B) Tumor measurements from three different experiments where Pmel and Pmel-Trx were transferred to 14 recipient mice, and combination of Pmel+Pmel−Trx were transferred to a total of 19 recipient mice is shown. (FIG. 6C) Compiled data from three separate experiments showing the number of mice that showed tumor growth or remained tumor free upon adoptive transfer of either Pmel, Pmel−Trx or mixture of Pmel and Pmel−Trx T cells. (FIG. 6D) Upper panel: Adoptively transferred T cells were tracked at day 24 and day 42 by staining for Thy1.1 (Pmel), and Thy1.2 (for Pmel−Trx) on TCR Vβ13+ gate. Lower panel: Cell surface expressing molecules CD62L/CD44 were determined after sequential gating on Vβ13 for gp100 TCR followed by staining for Pmel (Thy1.1; red arrow) and Pmel−Trx (Thy 1.2; blue arrow). Right panel: PBL from the experiment in left panel were stimulated with PMA/ionomycin for 4 hrs to stain intracellularly for IFN-γ after sequential gating on Vβ13+ T cells. (FIG. 6E) The tumor free animals were re-challenged with B16-F10 tumor, and the Vβ13 for gp100 TCR reactive cells were tracked by Thy1.1 (for Pmel) and Thy1.2 (for Pmel−Trx) staining (right panel). Quantitative determination of Vβ13+ T cells before and after tumor re-challenge is shown (left panel). N=3. ***p value<0.001 obtained using the slopes of tumor growth curves in linear regression model.

FIG. 7 depicts results from example experiments, demonstrating exogenous Trx renders increased anti-oxidant capacity, altered signaling and improved anti-tumor property to T cells. (FIG. 7A) Three day activated gp100 epitope reactive splenic T cells from Pmel and Pmel−Trx mouse were re-stimulated overnight with cognate antigen, and supernatant was evaluated for secreted Trx by ELISA as per protocol. N=3 **p<0.005. (FIG. 7B) Pmel T cells were labeled with CFSE and stimulated with cognate antigen gp100 for three days. The expression of Trx was then determined using flurochrome conjugated antibody by gating on the cells in different phase of division. (FIG. 7C) CFSE labeled Pmel T cells were stimulated with cognate antigen in absence or presence of rTRx at various doses. Pmel T cells activated with cognate antigen in presence of rTrx (5 μg/ml) were used at day 3 to determine the expression of Trx (FIG. 7D), iGSH (FIG. 7E), pAMPK (FIG. 7F), stemness genes using qPCR (FIG. 7G), Annexin V upregulation after 4 hr of TCR restimulation with cognate antigen hgp100 or non-cognate ova peptide (FIG. 7H), and Glucose uptake using 2NBDG (FIG. 7I). N=2-3. Numerical values=MFI. *p<0.05, **p<0.01. (FIG. 7J) Melanoma epitope gp100 reactive T cells obtained from Pmel TCR transgenic mouse were activated with cognate antigen either in presence or absence of recombinant thioredoxin (rTrx) for three days before being adoptively transferred to the immunocompetent C57BL/6 recipient mice with ten day subcutaneously established murine melanoma B16-F10. Tumor measurements from two different experiments where Pmel and Pmel+rTrx were transferred to 9 recipient mice was compiled for this presentation. ***p value<0.01 obtained at last time point of the tumor measurement before the experiment was terminated.

FIG. 8 depicts results from example experiments, demonstrating characterization of the human T cells transduced with tyrosinase reactive TIL1383I TCR construct with Trx gene insert. Human T cells from normal healthy donor peripheral blood were retrovirally transduced with TIL1383I+Trx vector (FIG. 8A) or TIL1383I vector, and characterized for: (FIG. 8B) transduction efficiency based on CD34 expression, (FIG. 8C) CD62L, CD44, CD45RA, CD45RO expression, and (FIG. 8D) Glucose uptake using 2NBDG assay. Cells were restimulated overnight with cognate hTyr peptide antigen using T2 cells for determining: (FIG. 8E) Mitochondrial membrane potential using TMRM, and (FIG. 8F) NO secretion using DAF. (FIG. 8G) RNA from TCR transduced cells after REP was used to determine expression of ‘stemness’ genes using qPCR. N=2. *p value<0.05; **p value<0.01, ***p value<0.005.

FIG. 9 depicts results from example experiments, demonstrating the Pmel−Trx T cells exhibited higher metabolites related to the pentose phosphate pathway (PPP) and tricarboxylic acid (TCA) cycle.

FIG. 10 depicts results from example experiments, demonstrating α-ketoglutarate (KG) pretreatment renders Tscm phenotype to T cells. Splenic T cells from C57BL/6 mouse were activated with anti-CD3/28 (each 2 μg/ml) either in absence or presence of α-KG for three days, after which: (FIG. 10A) Glucose uptake was determined using 2NBDG, and (FIG. 10B) Tscm phenotype was evaluated using CD62L and CD44 cell surface expression. N=2.

FIG. 11 depicts results from example experiments, demonstrating IL4 pretreated T cells show improved persistence in vivo. Splenic T cells from C57BL/6 mice on Thy1.1 or Thy1.2 background were activated for three days either in 1L2 or IL2+IL4 respectively, and mixed in 1:1 ratio before injecting i.v. in Rag1−/− recipients to determine any differences in homeostatic proliferation. Transferred T cells were tracked at day 28 by staining Thy1.1 and Th1.2 cells in various lymphoid and non-lymphoid tissues.

FIG. 12 depicts results from example experiments, demonstrating an example of human T cell transduction using TIL1383I TCR. (FIG. 12A). Transduction efficiency as measured by CD34 staining, as retroviral construct is tagged with truncated CD34. (FIG. 12B). Recognition of HLA-A2 human melanoma by TIL1383I TCR transduced human T cells.

FIG. 13 depicts results from example experiments, demonstrating high cell surface thiol expression correlates with stem-cell memory phenotype in T cells (Tscm). (FIG. 13A) Murine splenic T cells were stained with malemide dye to determine the c-SH expression, and gates for differences in c-SH expression were put arbitrarily to evaluate for Tscm phenotype by determining CD122 and Sca1 expression on CD44loCD62L+ gated cells. (FIG. 13B) Antigen activated gp100 TCR specific splenic T cells from Pmel and Pmel−Trx mouse were gated on CD44loCD62L+ and evaluated for CD122 and Sca1 expression. (FIG. 13C) RNA prepared from activated Pmel and Pmel−Trx T cells was used to determine expression of stem cell related genes. N=3.

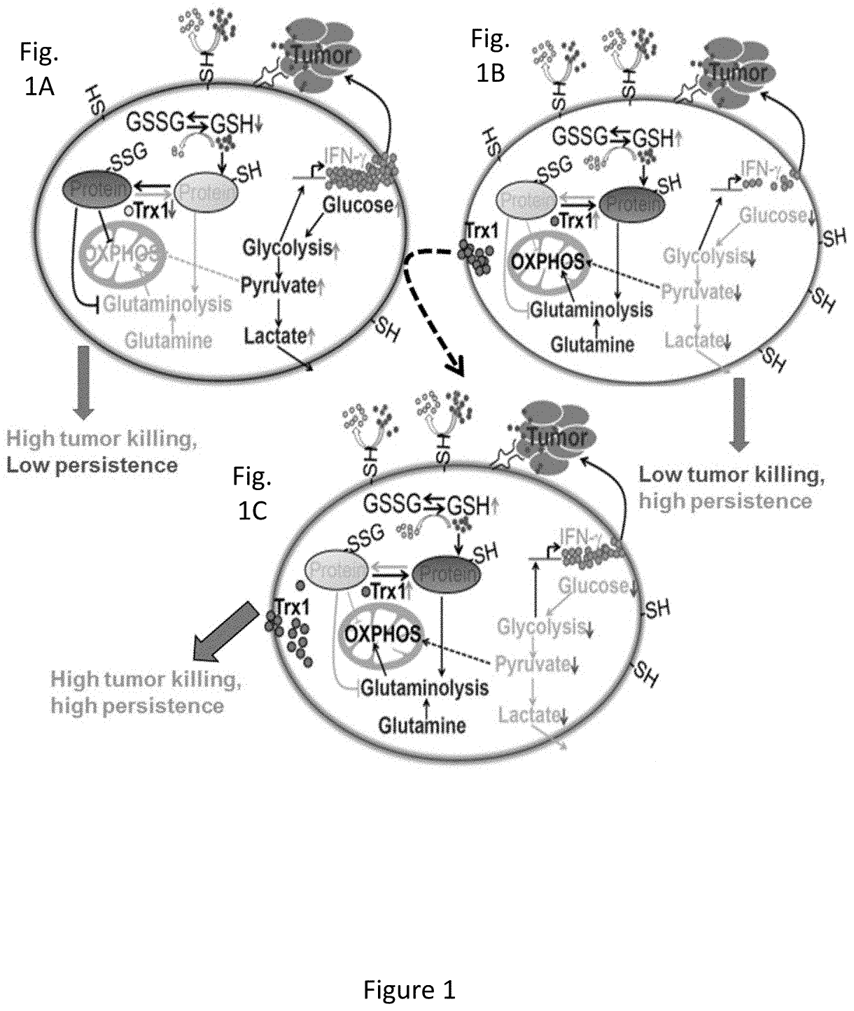

### DETAILED DESCRIPTION

Adoptive transfer of long-lived, multipotent memory T cells can improve persistence and enhance therapeutic efficacy of adoptive immunotherapies. The results presented herein demonstrate that cell surface thiols (c-SH) expression on T cells correlates with the development of T memory stem cells (Tscm) that persist longer in vivo and exhibit superior anti-tumor effector cells.

The invention relates to compositions and methods for treating cancer, including, but not limited to, hematologic malignancies and solid tumors. The present invention relates to a strategy of adoptive cell transfer of T cells generated by the reprogramming methods of the invention, whereby high cell surface thiols on T cells improve anti-tumor activity. In one embodiment, the desired T cell population is generated by increasing the level of thioredoxin (Trx) in T cells to enhance anti-tumor function of the T cell. In one embodiment, the desired T cell population is generated by treating T cells with recombinant thioredoxin (rTrx) to enhance anti-tumor function of the T cell.

In one embodiment, the invention provides compositions and methods to alter the redox status of T cells leading to their reprograming and generation of improved functional anti-tumor memory T cells. In one embodiment, Trx can regulate redox status and Tscm development, which in turn improves T cell persistence and function in vivo. The improved T cell persistence and function allows the use of the cells of the invention in more effective cancer treatments.

In one embodiment, the invention provides a method of improving immunity against melanoma cells. However, the invention should not be limited to melanoma. Rather, the invention is applicable to any disease, condition, or tumor associated with high oxidation levels such as lung cancer, ovarian cancer, and head and neck cancers.

In one embodiment, the invention provides compositions and methods to use thiols and antioxidant capacity as a biomarker for identifying T cells that not only persist longer in vivo, but also contribute to the generation of antitumor memory response in order to render long-term control of tumor growth.

In one embodiment, the invention provides compositions and methods to generate, expand, and enable redirection of memory T cells against cancer. In one embodiment, the invention provides a redox based strategy to promote higher levels of thiols by using Trx over expressing T cells (e.g., Trx T cells) to generate a therapeutic population of T cells for adoptive T cell therapy. In one embodiment, the invention provides a redox based strategy to promote higher levels of thiols by treating T cells with rTrx for adoptive T cell therapy.

In one embodiment, the invention provides compositions and methods to regulate anti-oxidant thiols and thiol signaling in a population of cells in order to modulate effector function and survival of tumor reactive T cells.

In one embodiment, the invention provides compositions and methods for generating T cells with high thiols (i.e. c-SH^{hi}) which harbors maximum Tscm cells. In one embodiment, the methods of the invention provide a strategy for the use of naturally occurring Tscm cells without any need for ex vivo programming to obtain the desirable cellular population for adoptive T cell therapy.

In one embodiment, the invention provides compositions and methods for co-injecting 1) tumor epitope reactive T cells with 2) tumor epitope reactive T cell over expressing thiol regulating anti-oxidant molecule Trx, for the generation of anti-tumor T cell memory that is functionally active and controls tumor growth upon tumor re-challenge in vivo.

In one embodiment, an advantage of the present invention is that the methods do not involve using high numbers of effector T cells because the invention is based on the generation of memory compartment that expands itself upon tumor re-challenge. In some instances, the adoptively transferred T cells of the present invention do not get exhausted, but rather the synergy between a tumor epitope reactive T cell (e.g., designated as T) with a tumor epitope reactive T cell over expressing thiol regulating anti-oxidant molecule (e.g., designated as TTrx) results in the ability to expand antigen re-challenge in vivo.

In one embodiment, the invention provides creating a reductive cellular environment by way of the pentose phosphate pathway (PPP) to promote T cell long-term survival, function, and a memory response.

In one embodiment, the invention provides the modulation of intracellular metabolites, such as alpha-ketoglutarate that are formed during metabolic commitment after T cell activation, to regulate T cell life-span and anti-tumor function.

In one embodiment, the invention provides compositions and methods for reprogramming T cells transduced with tumor reactive TCR by including thioredoxin over-expression in the TCR construct or by culturing ex vivo with alpha-ketoglutarate. In one embodiment, the invention provides compositions and methods for modulating T cells by treating the T cells with rTrx.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20%, ±10%, ±5%, ±1%, or ±0.1% from the specified value, as such variations are appropriate to perform the disclosed methods.

“Activation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antibody” as used herein, refers to an immunoglobulin molecule, which is able to specifically bind to a specific epitope on an antigen. Antibodies can be intact immunoglobulins derived from natural sources or from recombinant sources and can be immunoactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies and humanized antibodies (Harlow et al., 1988; Houston et al., 1988; Bird et al., 1988).

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, melanoma, lung cancer and the like.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

The term “fusion polypeptide” refers to a chimeric protein containing a protein of interest (e.g., luciferase) joined to a heterologous sequence (e.g., a non-luciferase amino acid or protein).

The term “homology” refers to a degree of complementarity. There may be partial homology or complete homology (i.e., identity). Homology is often measured using sequence analysis software (e.g., Sequence Analysis Software Package of the Genetics Computer Group. University of Wisconsin Biotechnology Center. 1710 University Avenue. Madison, Wis. 53705). Such software matches similar sequences by assigning degrees of homology to various substitutions, deletions, insertions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

The term “isolated” when used in relation to a nucleic acid, as in “isolated oligonucleotide” or “isolated polynucleotide” refers to a nucleic acid sequence that is identified and separated from at least one contaminant with which it is ordinarily associated in its source. Thus, an isolated nucleic acid is present in a form or setting that is different from that in which it is found in nature. In contrast, non-isolated nucleic acids (e.g., DNA and RNA) are found in the state they exist in nature. For example, a given DNA sequence (e.g., a gene) is found on the host cell chromosome in proximity to neighboring genes; RNA sequences (e.g., a specific mRNA sequence encoding a specific protein), are found in the cell as a mixture with numerous other mRNAs that encode a multitude of proteins. However, isolated nucleic acid includes, by way of example, such nucleic acid in cells ordinarily expressing that nucleic acid where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide contains at a minimum, the sense or coding strand (i.e., the oligonucleotide may be single-stranded), but may contain both the sense and anti-sense strands (i.e., the oligonucleotide may be double-stranded).

The term “isolated” when used in relation to a polypeptide, as in “isolated protein” or “isolated polypeptide” refers to a polypeptide that is identified and separated from at least one contaminant with which it is ordinarily associated in its source. Thus, an isolated polypeptide is present in a form or setting that is different from that in which it is found in nature. In contrast, non-isolated polypeptides (e.g., proteins and enzymes) are found in the state they exist in nature.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

By “expression cassette” is meant a nucleic acid molecule comprising a coding sequence operably linked to promoter/regulatory sequences necessary for transcription and, optionally, translation of the coding sequence.

The term “operably linked” as used herein refer to the linkage of nucleic acid sequences in such a manner that a nucleic acid molecule capable of directing the transcription of a given gene and/or the synthesis of a desired protein molecule is produced. The term also refers to the linkage of sequences encoding amino acids in such a manner that a functional (e.g., enzymatically active, capable of binding to a binding partner, capable of inhibiting, etc.) protein or polypeptide is produced.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a n inducible manner.

An “inducible” promoter is a nucleotide sequence which, when operably linked with a polynucleotide which encodes or specifies a gene product, causes the gene product to be produced substantially only when an inducer which corresponds to the promoter is present.

A “constitutive” promoter is a nucleotide sequence which, when operably linked with a polynucleotide which encodes or specifies a gene product, causes the gene product to be produced in a cell under most or all physiological conditions of the cell.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

As used herein, a “peptidomimetic” is a compound containing non-peptidic structural elements that is capable of mimicking the biological action of a parent peptide. A peptidomimetic may or may not comprise peptide bonds.

A “polynucleotide” means a single strand or parallel and anti-parallel strands of a nucleic acid. Thus, a polynucleotide may be either a single-stranded or a double-stranded nucleic acid. In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

The term “recombinant polypeptide” as used herein is defined as a polypeptide produced by using recombinant DNA methods. A host cell that comprises a recombinant polynucleotide is referred to as a “recombinant host cell.” A gene which is expressed in a recombinant host cell wherein the gene comprises a recombinant polynucleotide, produces a “recombinant polypeptide.”

As used herein, a “recombinant cell” is a host cell that comprises a recombinant polynucleotide.

By the term “specifically binds,” as used herein, is meant a molecule, such as an antibody, which recognizes and binds to another molecule or feature, but does not substantially recognize or bind other molecules or features in a sample.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, a “marker gene” or “reporter gene” is a gene that imparts a distinct phenotype to cells expressing the gene and thus permits cells having the gene to be distinguished from cells that do not have the gene. Such genes may encode either a selectable or screenable marker, depending on whether the marker confers a trait which one can ‘select’ for by chemical means, i.e., through the use of a selective agent (e.g., a herbicide, antibiotic, or the like), or whether it is simply a “reporter” trait that one can identify through observation or testing, i.e., by ‘screening’. Elements of the present disclosure are exemplified in detail through the use of particular marker genes. Of course, many examples of suitable marker genes or reporter genes are known to the art and can be employed in the practice of the invention. Therefore, it will be understood that the following discussion is exemplary rather than exhaustive. In light of the techniques disclosed herein and the general recombinant techniques which are known in the art, the present invention renders possible the alteration of any gene.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “therapeutic” as used herein means a treatment and/or prophylaxis. A therapeutic effect is obtained by suppression, remission, or eradication of a disease state.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention provides compositions and methods for treating cancer as well as other diseases. The cancer may be a hematological malignancy, a solid tumor, a primary or a metastasizing tumor. Other diseases treatable using the compositions and methods of the invention include viral, bacterial and parasitic infections as well as autoimmune diseases.

Adoptive transfer of the cells of the invention is an attractive therapy for the treatment of cancer. In one embodiment, the invention provides a cell that has been modified to exhibit increased expression of cell surface thiols (c-SH). In one embodiment, promoting high expression of c-SH on T cells allows for reprogramming the T cell towards a memory stem cells (Tscm) phenotype with improved anti-tumor activity. In one embodiment, the reprogramming is achieved using genetic means. In another embodiment, the reprogramming is achieved using biochemical means. In one embodiment, high expression of c-SH (c-SH^{hi}) is a marker for Tscm cells.

In one embodiment, the invention provides any means to increase c-SH on T cells. For example, c-SH^{hi} T cells can be generated by way of genetic modification (Trx gene transfer) and pharmacologic modulators (e.g., IL-4, rTrx, rTrx-reductase, glutathione, α-ketoglutarate, proline).

In one embodiment, the high anti-oxidant capacity of c-SH^{hi} T cells provide the reductive niche advantageous to promote the generation of anti-tumor memory in an otherwise suppressive oxidative tumor microenvironment.

In one embodiment, the invention provides co-culturing or co-injection of a first antigen reactive T cell (e.g., designated as T) with a second antigen reactive T cell overexpressing a thiol regulating anti-oxidant molecule (e.g., Trx) to generate anti-tumor Tscm phenotype cells. For example, the combination of the first and second T cell types is responsible for induction of the memory phenotype. In one embodiment, the level of thiol/thioredoxin on the surface of T cells regulates the generation of tumor reactive memory T cells in vivo. In one embodiment, the invention provides co-injection of a first antigen reactive T cell with a second antigen reactive T cell that has been exposed to a thiol regulating anti-oxidant molecule (e.g., Trx) to generate anti-tumor Tscm phenotype cells.

In one embodiment, the invention provides a cell population having anti-tumor activity and in vivo persistence of these cells provides advantages for use in adoptive T cell therapy.

### Antigen

In one embodiment, the invention provides generating anti-tumor Tscm phenotype cells. The cells can be generated to be reactive to any desirable tumor antigen of interest. In the context of the present invention, “tumor antigen” or “hyperproliferative disorder antigen” or “antigen associated with a hyperproliferative disorder,” refers to antigens that are common to specific hyperproliferative disorders such as cancer. The antigens discussed herein are merely included by way of example. The list is not intended to be exclusive and further examples will be readily apparent to those of skill in the art.

Tumor antigens are proteins that are produced by tumor cells that elicit an immune response. The selection of the antigen binding domain of the invention will depend on the particular type of cancer to be treated. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In one embodiment, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen referred to in the invention may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

Depending on the desired antigen to be targeted, the cells of the invention can be modified to target the appropriate antigen.

### Vectors

The present invention encompasses a genetic means to increase c-SH on T cells. For example, the nucleic acid sequences coding for the desired molecule (e.g., Trx, genes that increase the expression of Trx, or genes that inhibit the negative regulators of Trx) can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In brief summary, the expression of natural or synthetic nucleic acids is typically achieved by operably linking a nucleic acid encoding the desired polypeptide or portions thereof to a promoter, and incorporating the construct into an expression vector. The vectors can be suitable for replication and integration in eukaryotes. Typical cloning vectors comprise transcription and translation terminators, initiation sequences, and promoters useful for regulation of the expression of the desired nucleic acid sequence.

The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, and 5,589,466, incorporated by reference herein in their entireties. In another embodiment, the invention provides a gene therapy vector.

The nucleic acid can be cloned into a number of different types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector comprises an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

### Sources of T Cells

Prior to expansion, a source of T cells is obtained from a subject. Examples of subjects include humans, dogs, cats, mice, rats, and transgenic species thereof. Preferably, the subject is a human. T cells can be obtained from a number of sources, including peripheral blood mononuclear cells, bone marrow, lymph node tissue, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many or all divalent cations. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood by lysing the red blood cells and depleting the monocytes, for example, by centrifugation through a PERCOLL™ gradient. Alternatively, T cells can be isolated from umbilical cord. In any event, a specific subpopulation of T cells can be further isolated by positive or negative selection techniques.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations.

### Therapeutic Application

In one embodiment, the present invention includes a type of cellular therapy where T cells are modified to exhibit high expression of c-SH and the cell can be infused to a recipient in need thereof. The infused cell is able to kill tumor cells in the recipient. Unlike antibody therapies, the T cells of the invention are able to result in long-term persistence that can lead to sustained tumor control. In another embodiment, the present invention includes a type of cellular therapy wherein T cells are modified by treating the T cells with an agent, such as rTrx, and the T cells can then be infused to a recipient in need thereof. The infused cell is able to kill tumor cells in the recipient.

In one embodiment, the T cells of the invention exhibiting high anti-oxidant capacity are able to promote differentiation and maintenance of memory T cells in oxidative tumor microenvironments. In one embodiment, the cells of the invention are able to generate anti-tumor Tscm phenotype cells in vivo.

In another embodiment, the T cells of the invention evolve into specific memory T cells that can be reactivated to inhibit any additional tumor formation or growth. For example, the cells of the invention exhibit persistence and increased anti-tumor activity. Without wishing to be bound by any particular theory, T cells of the invention may differentiate in vivo into a central memory-like state upon encounter and subsequent elimination of target cells expressing the surrogate antigen.

Without wishing to be bound by any particular theory, the anti-tumor immunity response elicited by the T cells of the invention may be an active or a passive immune response. In addition, the immune response may be part of an adoptive immunotherapy approach in which T cells induce an immune response specific to a desired antigen.

Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the CARs of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, meningioma, neuroblastoma, retinoblastoma and brain metastases).

The T cells of the invention may also serve as a type of vaccine for ex vivo immunization and/or in vivo therapy in a mammal. Preferably, the mammal is a human.

Ex vivo procedures are well known in the art and are discussed more fully below. Briefly, cells are isolated from a mammal (preferably a human) and genetically (i.e., transduced or transfected in vitro) or biochemically (i.e., treated with an agent, such as rTrx) modified. The modified cell can be administered to a mammalian recipient to provide a therapeutic benefit. The mammalian recipient may be a human and the modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present invention also provides compositions and methods for in vivo immunization to elicit an immune response directed against an antigen in a patient.

### Pharmaceutical

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “a tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 104 to 109 cells/kg body weight, preferably 105 to 106 cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol may serve to select out certain populations of T cells.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the T cell compositions of the present invention are administered to a patient by intradermal or subcutaneous injection. In another embodiment, the T cell compositions of the present invention are preferably administered by i.v. injection. The compositions of T cells may be injected directly into a tumor, lymph node, or site of infection.

### Combinations

In one embodiment, the composition of the present invention comprises a combination of modulators described herein. For example, in one embodiment the composition comprises a reprogrammed T cell in combination with an agent that increases the anti-cancer effects of the composition. In certain embodiments, a composition comprising a combination of modulators described herein has an additive effect, wherein the overall effect of the combination is approximately equal to the sum of the effects of each individual inhibitor. In other embodiments, a composition comprising a combination of modulators described herein has a synergistic effect, wherein the overall effect of the combination is greater than the sum of the effects of each individual modulator.

The reprogrammed T cells of the present invention may be co-administered to a subject with any cancer treatment known in the art.

In one embodiment, the subject is treated with reprogrammed T cells and an antiproliferative agent. Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

In one embodiment, the subject is treated with reprogrammed T cells and a chemotherapeutic agent. Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

In one embodiment, the subject is treated with reprogrammed T cells and another anti-tumor agent, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine. Anti-angiogenic agents are well known to those of skill in the art. Suitable anti-angiogenic agents for use in the methods and reprogrammed T cells of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the reprogrammed T cells of the invention include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin IL, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Increased Expression of Anti-Oxidant Molecule Thioredoxin-1 Immuno-Metabolically Alters Anti-Tumor T Cells and Potentiates Tumor Control

Adoptive transfer of tumor epitope reactive T cells is a promising strategy to control tumor growth. However, chronically stimulated T cells expanded for adoptive cell transfer (ACT) are susceptible to cell death in an oxidative tumor microenvironment. Without wishing to be bound by any particular theory, since oxidation of cell surface thiols (c-SH) also alters functionality of proteins, it was hypothesized that increased level of thioredoxin, an anti-oxidant molecule that facilitates reduction of proteins by cysteine thiol-disulfide exchange, in T cells will result in sustained anti-tumor function. Therefore, Trx1 transgenic mice were crossed with gp100 reactive TCR (Pmel) to generate Pmel/Trx mice. The Trx overexpressing transgenic T cells expressed higher thiols that inversely correlated with ROS, and susceptibility to TCR restimulation or H₂O₂ mediated cell death. These Trx1 expressing T cells showed CD62L^{hi} central memory-like (Tcm) phenotype with reduced effector function (IFNγ^{lo}2-NBDG^{lo}). However, adoptive co-transfer of Pmel and Pmel−Trx T cells resulted in vastly improved tumor control than was observed with Pmel or Pmel−Trx alone. Additionally, using tumor reactive T cells cultured in presence of recombinant Trx (rTrx) also resulted in better persistence and improved tumor control. Importantly, Trx expression or including Trx in culture conditions resulted in increased dependence of T cells on mitochondrial metabolism and a unique metabolite signature conducive to tumor control. Thus, strategies to increase anti-oxidant capacity of anti-tumor T cells modulate its immune-metabolic phenotype, leading to immunotherapeutic control of tumors.

Trx Transgenic T Cells Exhibit Increased c-SH and iGSH Reduced Susceptibility to Tumor Microenvironment (TME) Mediated Oxidative Stress

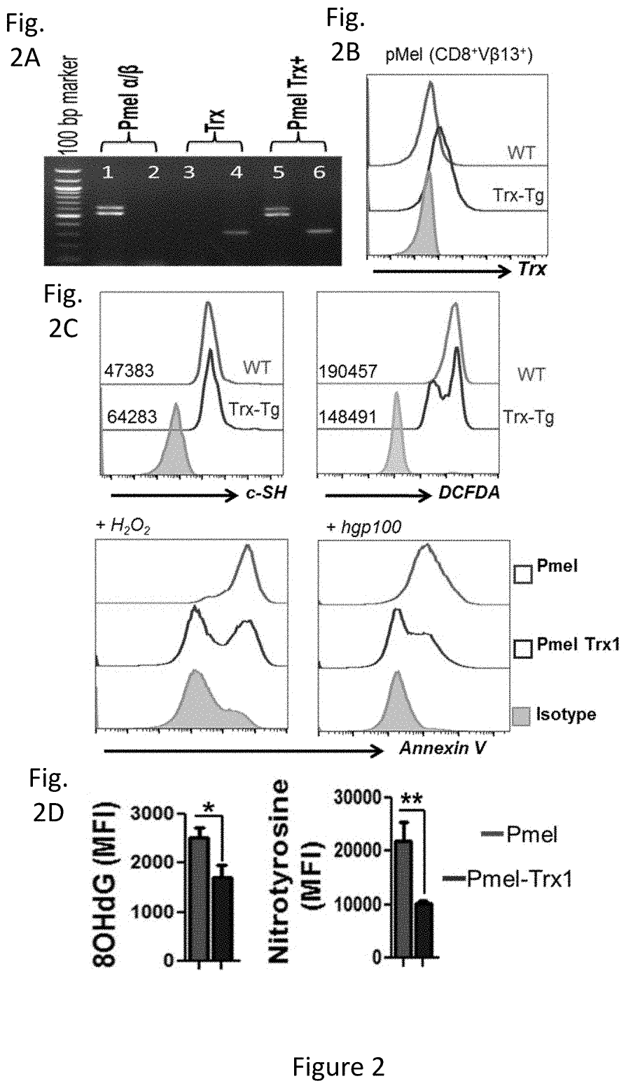

Trx is a class of 12 kDa ubiquitous redox proteins found primarily in the cytosol (Sengupta R et al., 2014, World J Biol Chem, 5(1):68-74). Trx possess a catalytically active di-thiol function in a Cys-Gly-Pro-Cys motif and are present in all organisms. Biomolecules with redox-active sulfhydryl function(s), (thiol —SH compounds), are necessary for the maintenance of mildly reductive cellular environments to counteract oxidative stress, and for the execution of redox reactions for metabolism and detoxification (Dickinson D A et al., 2002, Biochem Pharmacol, 64(5-6):1019-26; Mailloux R J et al., 2014, Redox Biol, 2:123-39). Melanoma epitope gp100 reactive TCR bearing transgenic mouse Pmel was bred with Thioredoxin1 (Trx1)-transgenic mouse, in which human Trx1 is systemically over expressed under control of the β-actin promoter (Adluri R S et al., 2011, J Mol Cell Cardiol, 50(1):239-47), to obtain Pmel−Trx mouse. FIG. 2A shows successful generation of the Pmel−Trx mice. The gel picture shows the characterization of the Pmel−Trx mice. While Pmel mice showed gp100 TCRα (600 bp) and TCRβ (500 bp) in lane 1, Trx expression was undetectable in lane 2. Similarly, the Trx-Tg mouse does not show any Pmel TCRαβ expression (lane 3), but is positive for Trx (lane 4). Further, the Pmel−Trx mouse shows expression of Pmel TCRαβ (lane 5), and Trx (lane 6). The FACS plot (FIG. 2B) shows a FACS based comparison for Trx expression on gp100 reactive T cells from Pmel vs. Pmel−Trx mice. FIG. 2C shows increased expression of cell surface thiols (c-SH) in splenic Pmel−Trx T cells as compared to T cells from Pmel mice (left panel). The c-SH staining was done using the alexa-fluor labeled maleimide dye (Invitrogen), as previously reported (Gelderman K A et al., 2006, Proc Natl Acad Sci USA, 103(34):12831-6; Sebastia J et al., 2003, Cytometry A, 51(1):16-25). This increased anti-oxidant thiol levels inversely correlated to the ROS levels in the activated T cells as measured by DCFDA (upper right), and less cell death (mediated by both H₂O₂ and TCR restimulation) (lower panel).

In order to confirm the functional advantage of Trx overexpression on T cells, activated congenic Pmel or Pmel−Trx Tg T cells were transferred i.p. into the C57BL/6 mice with EL4 ascites. The analysis of Vβ13+ T cells retrieved after 24 hr. from ascites showed reduced 8-hydroxy guanine (8-OHdG), and reduced nitrotyrosine (marker for ROS/RNS stress) (Valavanidis A et al., 2009, J Environ Sci Health C Environ Carcinog Ecotoxicol Rev, 27(2):120-39; Darwish R S et al., 2007, J Trauma, 63(2):439-42; Nagaraj S et al., 2007, Nat Med, 13(7):828-35) in Pmel−Trx, as compared to Pmel cells alone (FIG. 2D). This established that increased Trx levels indeed protect the T cells in oxidative TME.

### Increased Trx Expression Alters T Cell Signaling

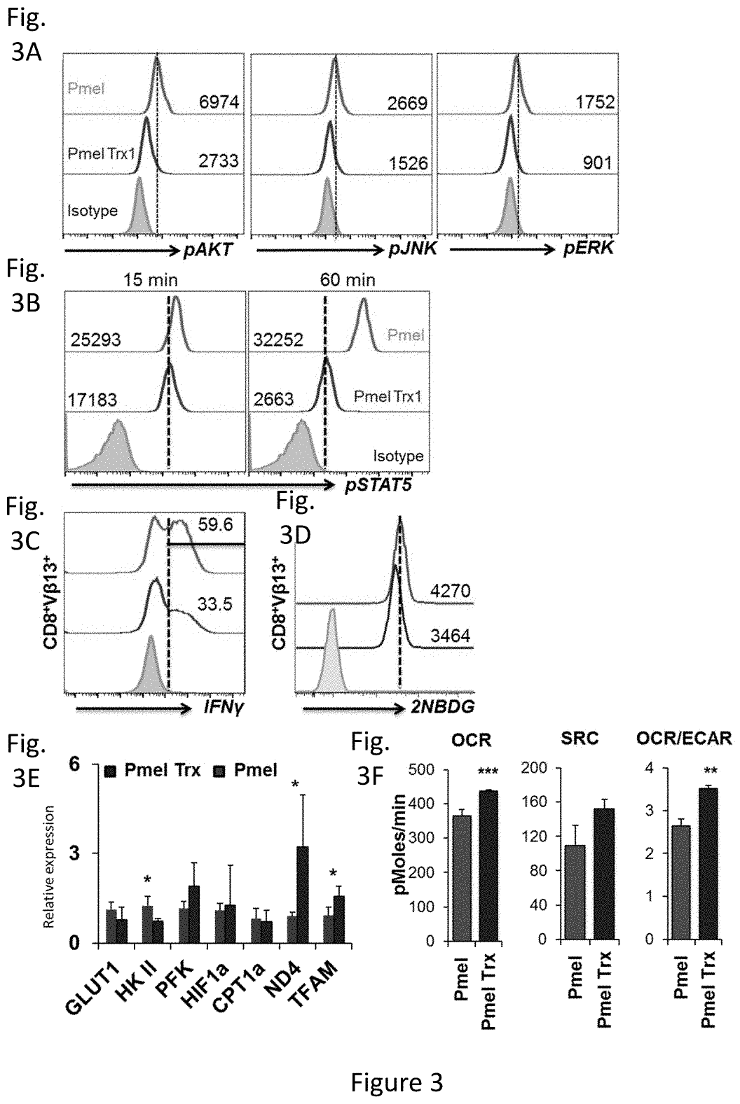

To determine if any differences exist in TCR signaling between the Pmel and Pmel−Trx cells the phosphorylation levels of key signaling molecules AKT, JNK and STAT5 were determined. The data in FIG. 3A shows a reduced level of pAKT, pJNK, pERK. Given the importance of STAT5 involvement in assessing a T cell response to the cytokine microenvironment that shapes its function (Tripathi P et al., 2010, J Immunol, 185(4):2116-24), the pSTAT5 was determined in Pmel−Trx T cells. It was observed that Pmel−Trx T cells have reduced upregulation of pSTAT5 as compared to the Pmel T cells (FIG. 3B). This also corresponded to the reduced ability of Pmel−Trx T cells to secrete cytokine IFN-γ (33% by Pmel−Trx vs. 59% by Pmel) (FIG. 3C), which is shown to dependent upon glucose availability (Cham C M et al., 2005, J Immunol, 174(8):4670-7; Chang C H et al., 2013, Cell, 153(6):1239-51). Using fluorescence glucose substrate 2NBDG it was also observed that Pmel−Trx T cells exhibit lower glucose uptake as compared to the Pmel T cells alone (FIG. 3D). A real-time PCR analysis confirmed that activated Pmel−Trx T cells exhibit reduced expression of glucose transporter Glut1, and lesser expression of key glycolytic molecule hexokinase II (HKII) (FIG. 3E). On the contrary, the transcription factor associated with mitochondria (TFAM) and ND4 was observed to be upregulated in Pmel−Trx T cells. This indicated that the Trx overexpressing T cells may be more dependent upon mitochondrial oxidative phosphorylation, than glycolysis for their energy demands. A seahorse based metabolic flux analysis confirmed that antigen Pmel−Trx T cells activated with cognate antigen for three days exhibit higher oxygen consumption rate (OCR), and possessed enhanced spare respiratory capacity (SRC) compared to the Pmel T cells alone (FIG. 3F). The extracellular acidification rate (ECAR) was also found to be lower in Pmel−Trx as compared to Pmel T cells. Overall, these differences established that OCR/ECAR ratio was higher in Pmel−Trx as compared to Pmel (FIG. 3F, right panel). Without wishing to be bound by any particular theory, this suggests that Trx overexpression leads to increased dependence on mitochondrial oxidative phosphorylation as compared to glycolysis.

Comprehensive Metabolic Profiling of Activated Pmel Vs. Pmel−Trx Shows Distinct Metabolites in Pmel−Trx T Cells

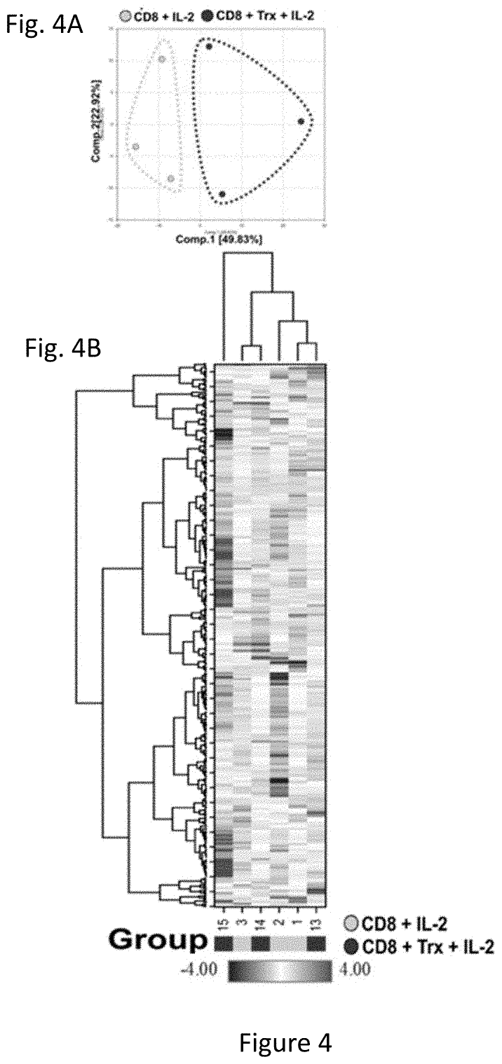

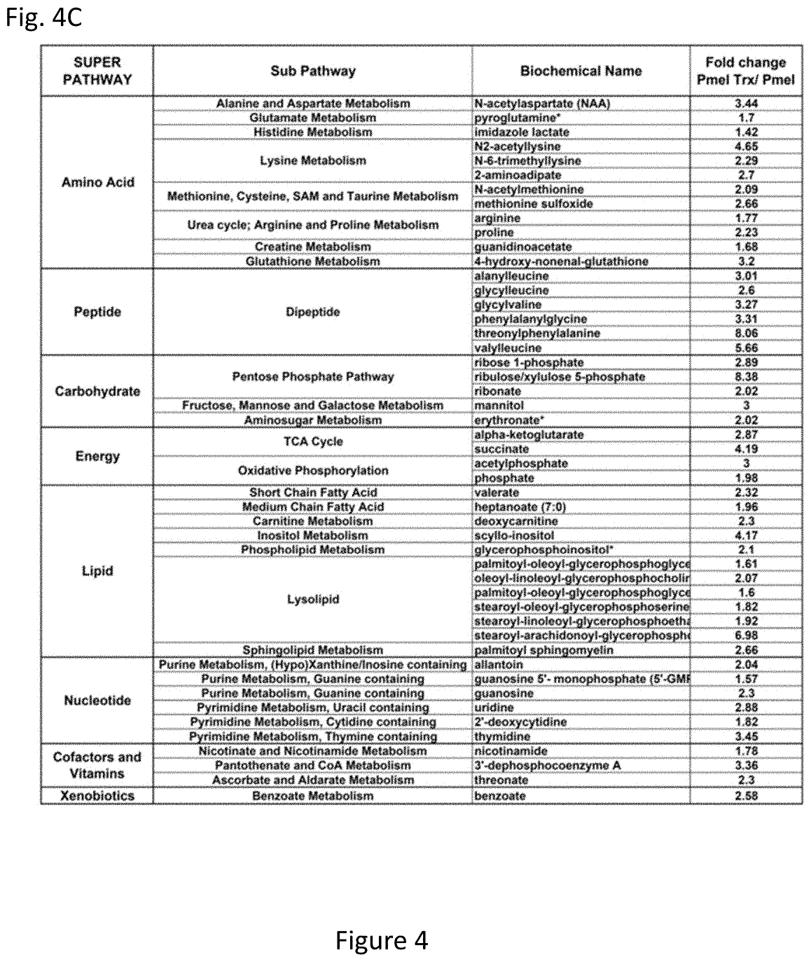

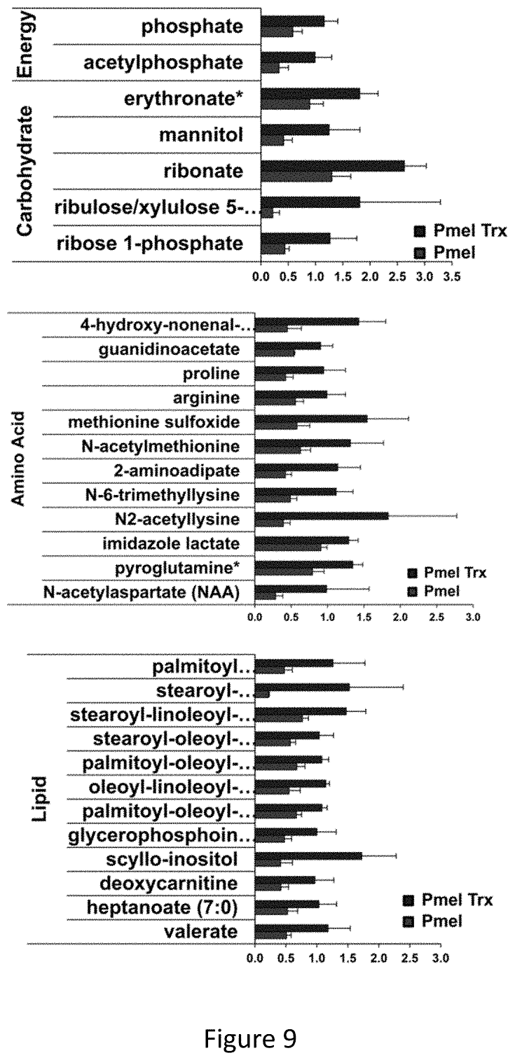

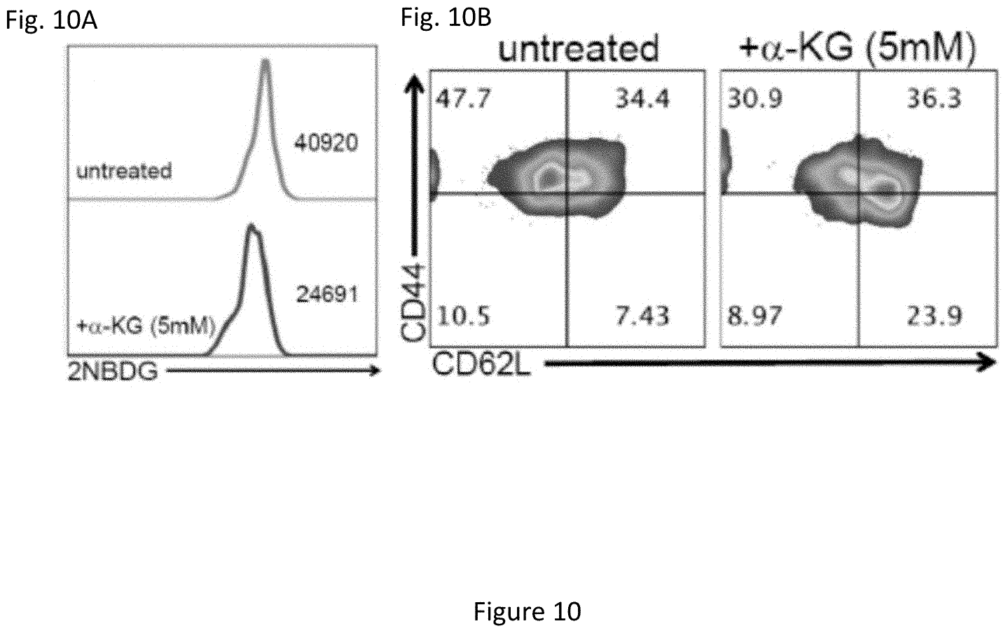

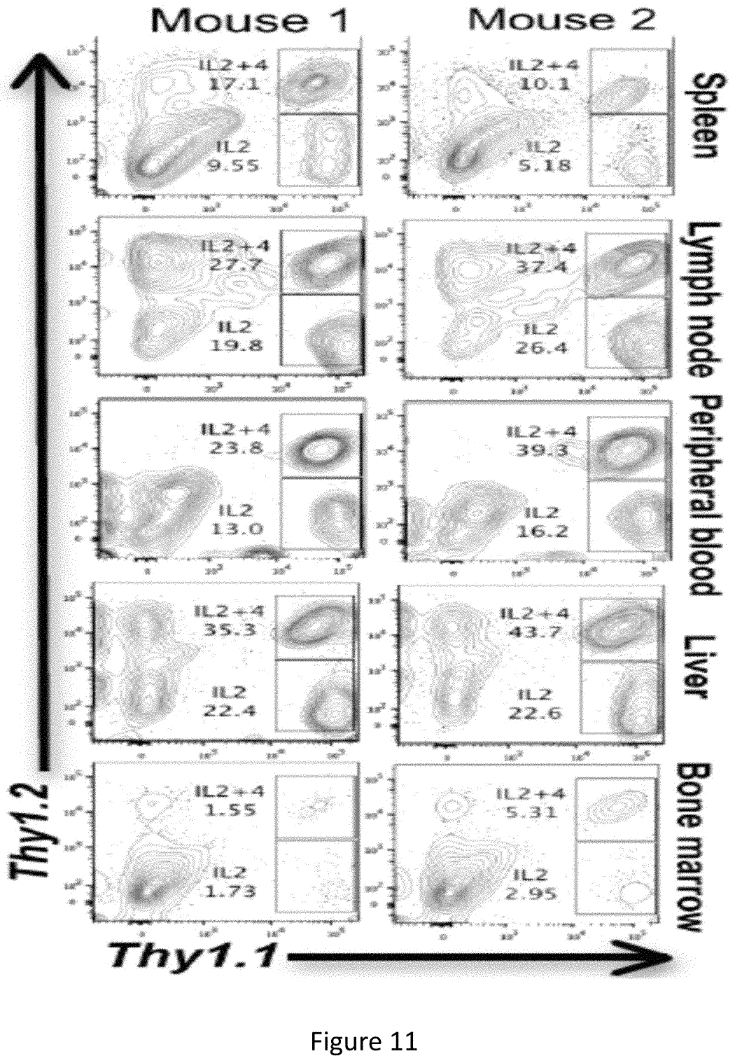

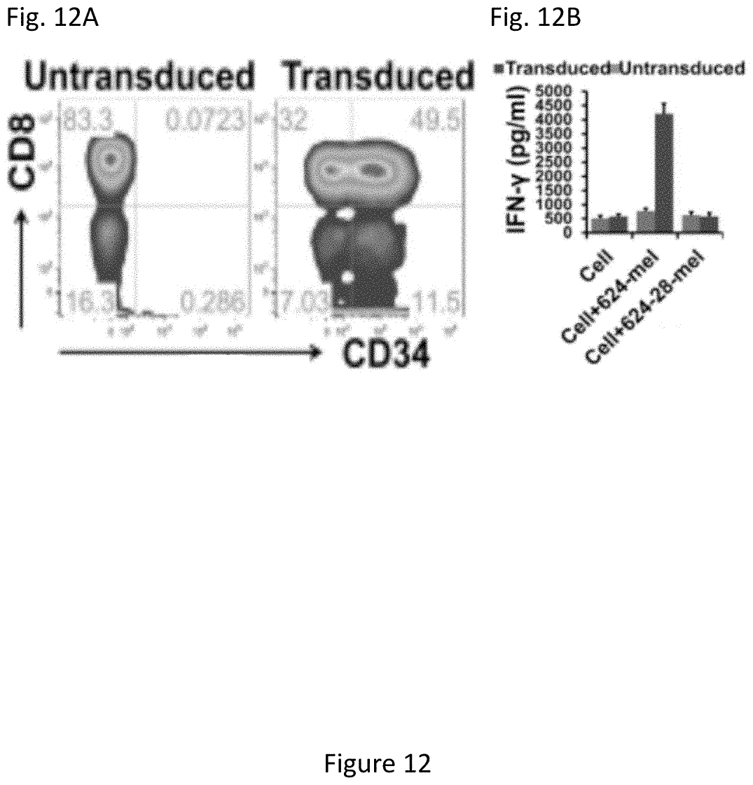

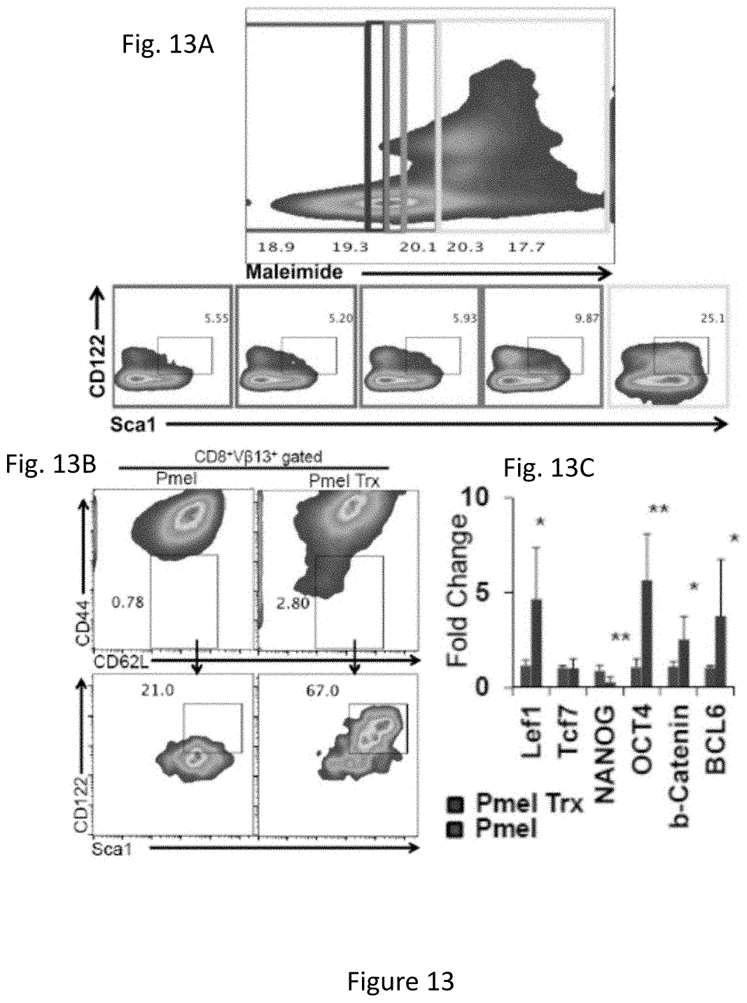

Since commitment of the T cells to different metabolic pathways has been shown to result in differential fate of the T cells (Sukumar M et al., 2013, J Clin Invest, 123(10):4479-88), the services of a commercial vendor Metabolon Inc. (NC) were utilized to quantify the differences in metabolites accumulated within the TCR activated Pmel vs. Pmel−Trx T cells. For this purpose 10 million activated Pmel and Pmel−Trx T cells were sorted and the pellets were frozen as per the protocol before overnight shipping, and analysis was done using the Gas Chromatography-Mass Spectroscopy (GC-MS). The principle component analysis in FIG. 4A summarizes the degree of differences between metabolites in Pmel vs. Pmel−Trx T cells, whereas FIG. 4B shows the hierarchically clustered heat map. Specifically, the Pmel−Trx T cells exhibited higher metabolites related to pentose phosphate pathway (PPP) and tricarboxylic acid (TCA) cycle (FIG. 4C, and FIG. 9). In addition, the level of fatty acids were also high in Trx cells, indicating that fatty acid oxidation could be the key source of energy for these cells (as is the case with memory T cells) (van der Windt G J, et al., 2012, Immunity, 36(1):68-78). While a number of amino acids were found to be upregulated in Pmel−Trx T cells, the noticeable were the ones that have been shown to be involved in life-span extension, i.e. serine, proline, or histidine (Edwards C et al., 2015, BMC Genet, 16(1):8). Importantly, Trx are characterized by the presence of three conserved prolines, with one located between the catalytic cysteine residues of the -Cys-Gly-Pro-Cys-motif. Proline is also the key residue that determines the reducing power of Trx and replacing it by a serine or a threonine has a dramatic effect on the redox and stability properties of the protein (Edwards C et al., 2015, BMC Genet, 16(1):8). Thus, without wishing to be bound by any particular theory, it is believed that the thioredoxin over expression in T cells potentiates the reductive phenotype. This could be attributed to increased usage of PPP pathway (as evident by increased NADPH), and accumulation of the alpha-ketoglutarate (α-KG). Both these processes may be a likely less dependent on TCA cycle or increased glutamine uptake. A recent study showed that α-KG blocks ATP synthase and lowers ATP levels in the cells with longer lifespan (Chin R M et al., 2014, Nature, 510(7505):397-401), and aids in stem cell differentiation (Ochocki J D et al., 2013, J Cell Biol, 203(1):23-33).

### Increased Glutamine Uptake by Trx Overexpressing T Cells Imprints Unique Matabolic Advantage

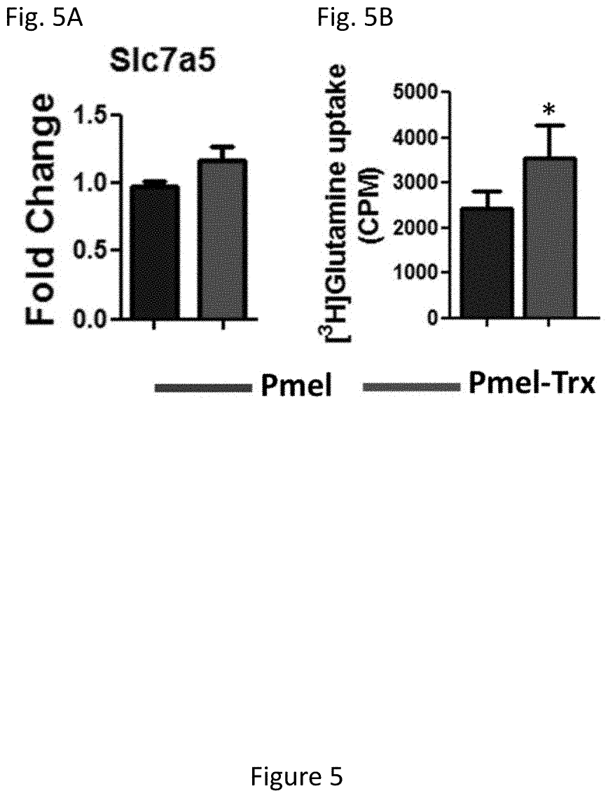

The observation that Trx-T cells exhibit elevated levels of metabolite α-KG, that is also replenished by anaplerotic reactions using glutamine and enters into the mitochondrial citric acid cycle, led to the hypothesis that differential glutamine levels in Trx overexpressing T cells may be responsible for their increased persistence in tumor microenvironment. It has also been shown that while glutamine helps differentiation of T cells to effector phenotype (Klysz D et al., 2015, Sci Signal, 8(396):ra97), the deficiency of glutamine can result in formation of Treg (Klysz D et al., 2015, Sci Signal, 8(396):ra97). To ascertain the role of glutamine in rendering T cell memory phenotype, the in vitro experiments where Pmel-T cells were activated with cognate antigen in presence of either recombinant Trx using different media conditions were initiated: a) glucose only (glutamine free), b) glutamine only (glucose free), and c) with glucose and glutamine. The activated T cells at 72 h were then evaluated for viability, cytokine (IFNγ, IL2) secretion, cell-surface molecule expression (CD95, CD95L, CD62L, CD44, Sca1, CD122, CCR7, c-SH, iGSH). The repeat experiments were performed to specifically establish if any changes observed in phenotype or function are glutamine catabolism specific by blocking glutaminolysis with 6-diazo-5-oxo-L-norleucine (DON), a glutaminase inhibitor (Curthoys N P et al., 1995, Annu Rev Nutr, 15:133-59). Since increased levels of glutamine leading to higher α-KG accumulation can be either due to increased activity of glutamine synthetase, or increased transportation of glutamine (due to transporters ASCT2, SNAT1, SNAT2) (Poffenberger M C et al., 2014, Immunity, 40(5):635-7), or higher degree of glutaminolysis (i.e. degradation of available glutamine), it was determined if the contribution of each of these pathways by determining the mRNA expression of these molecules (FIG. 5A). Further, to comprehensively establish the role of glutamine dependence of memory T cells, the tracer studies were used by incubating the FACS sorted memory T cells with L-(3,4-3H (N)) glutamine (0.5 mCi) for 5 min at room temperature, and incorporation per cell was measured as counts per minute (CPM) as detailed earlier (Klysz D et al., 2015, Sci Signal, 8(396):ra97). The data show that Pmel−Trx T cells have increased glutamine uptake as compared to the Pmel T cells (FIG. 5B).

### Pmel−Trx Cells Result in Sustained Tumor Control

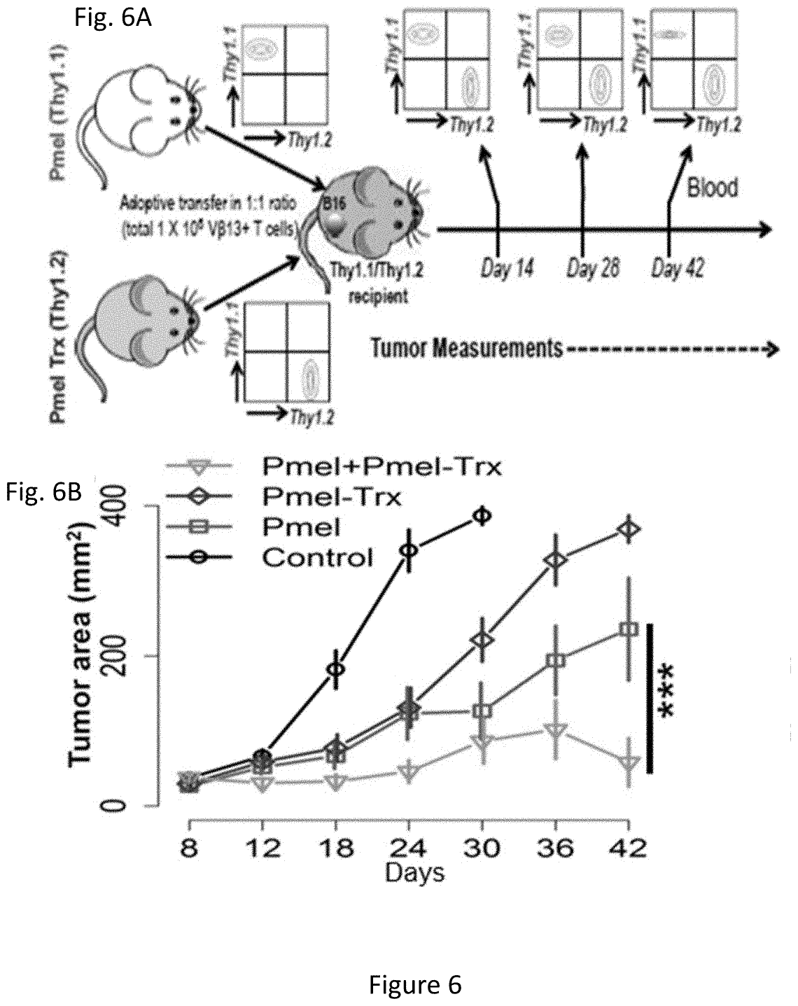

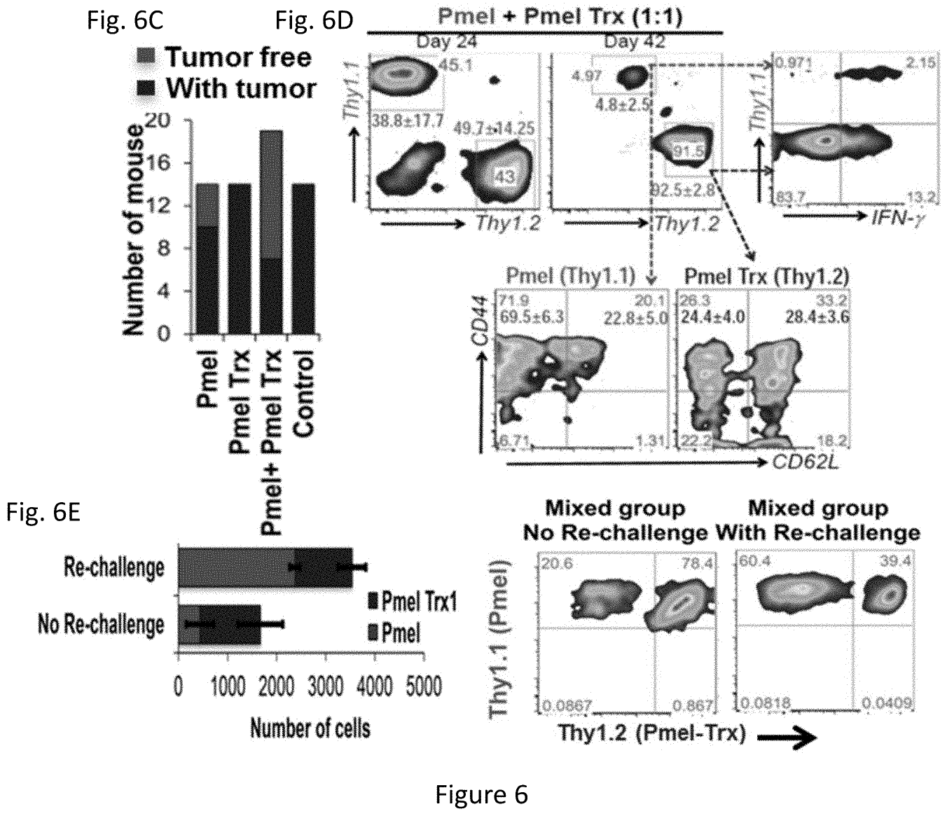

To compare the efficacy of the Pmel−Trx T cells in controlling tumors, 1×10⁶ Vβ13+ T cells from either Pmel or Pmel−Trx mice were transferred to different groups of C57BL/6 mice bearing subcutaneously established murine melanoma B16-F10. Given that stable over expression of Trx may inhibit in vivo conversion, (as shown by other studies for Tcm phenotype cells) (Sukumar M et al., 2013, J Clin Invest, 123(10):4479-88; Quezada S A et al., 2010, J Exp Med, 207(3):637-50; Xie Y et al., 2010, J Exp Med, 207(3):651-67), Pmel T cells and Pmel−Trx T cells were co-injected in 1:1 ratio to test if activated Pmel T cells (with immediate effector phenotype) will control the established tumor and the long-term persistence of the Pmel−Trx T cells will lead to subsequent control of any tumor growth (FIG. 6A). The ACT experiment showed that the Pmel−Trx T cells indeed exhibited better persistence as compared to Pmel-T cells alone, but the relative ability to control tumor was not much different than the Pmel T cells (FIG. 6B, blue curve vs. red curve). However, as hypothesized, co-injecting both Pmel and Pmel−Trx T cells resulted in tumor control in 50-70% of the mice (FIG. 6B, green curve and cumulative data in FIG. 6C, where red portion of the bar shows tumor free). Further tracking the co-injected Pmel and Pmel−Trx T cells on day 24 and 42 in the recipient mice, which showed tumor control, demonstrated that Pmel−Trx T cells were more in numbers as compared to the Pmel T cells at day 42 (FIG. 6D, upper panel dot plots). Further tracking of phenotype and function at day 42 showed that that the majority of Pmel−Trx (Thy1.2) T cells exhibited CD62L+CD44+ Tcm phenotype, and also had a significant fraction with Tscm like CD62L+CD44− phenotype (18.2%, FIG. 6D, lower right dot panel, blue arrow), whereas 70% of Pmel (Thy1.1) T cells exhibited CD62L-CD44+ Tem phenotype (FIG. 6D, lower left dot plot, red arrow). Ex vivo re-stimulation of these cells showed (FIG. 6D, upper extreme right panel) that almost all Pmel T cells (Thy1.1+, red arrow) secreted cytokine IFNγ, as compared to the Pmel−Trx T cells (Thy1.1−, blue arrow). However, most surprisingly when the tumor free animals from the co-injected group were re-challenged with B16-F10 tumors it was the Pmel T cells (Thy1.1) that proliferated and expanded by day 10 post-tumor challenge (FIG. 6E, red section of the bar on left panel, and upper left quadrant in right panel of dot plot), whereas the number of Pmel−Trx T cells remained almost unchanged (FIG. 6E, blue section of the bar). This data indicates that anti-tumor memory T cells were generated in vivo from co-injected Pmel T cells in presence of Pmel−Trx T cells. It is likely that after the immediate effector Pmel-T cells undergo AICD induced by tumor antigen mediated chronic TCR re-stimulation, the remaining memory T cell pool is protected by Pmel−Trx T cells that persist in oxidative tumor microenvironment to provide “anti-oxidant help” leading to suitable reductive niche.

Pmel−Trx T Cells Secreted Trx Upon Antigen Stimulation. And Recombinant Trx Reprograms Pmel T Cells Ex Vivo

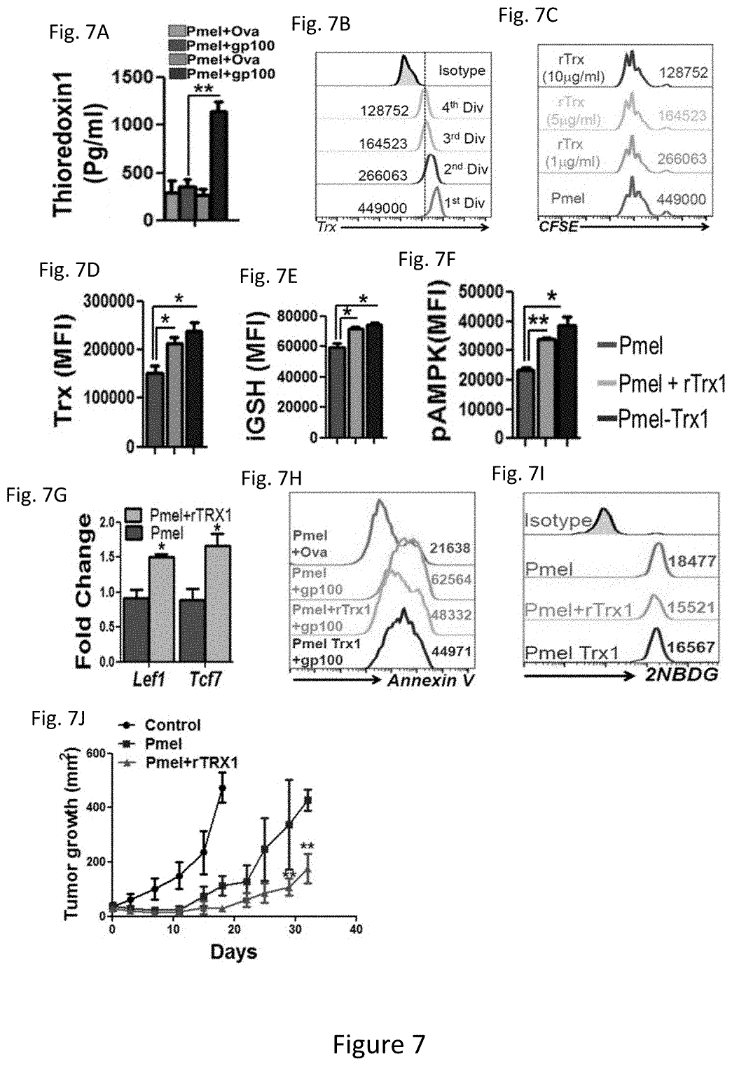

To get an insight into the mechanism that may be responsible for the ability of co-injected Pmel T cells to respond to tumor rechallenge (as in FIG. 6), it was determined if Pmel−Trx secreted Trx upon antigen restimulation. The data in FIG. 7A shows that as compared to Pmel T cells, Pmel−Trx T cells upon overnight activation with cognate antigen do secrete significantly high amount (≥1000 μg/ml) of Trx in the culture supernatant. Further, to determine how secreted Trx would have modulated the Pmel T cells, recombinant Trx was used during antigen induced in vitro Pmel activation and compared the phenotype with untreated Pmel and Pmel−Trx T cells. The data shows that T cells lose Trx with every cell division (FIG. 7B), as has been linked with cellular aging (Kesarwani P et al., 2014, Cancer Res, 74(21):6036-47; Yoshida T et al., 2003, Antioxid Redox Signal, 5(5):563-70), and incorporating rTrx during T cell activation is non-toxic as it does not hamper the cell division (FIG. 7C), but leads to increased expression of Trx and iGSH (FIG. 7D, FIG. 7E). It has also been shown recently that Trx1-mediated reduction of Cys130 and Cys174 is essential for AMPK function, and decreased Trx levels could lead to oxidation of Cys130 and Cys174 by inducing aggregation that prevents its activation and phosphorylation by AMPK kinases (Shao D et al., 2014, Cell Metab, 19(2):232-45). The data in FIG. 7F shows that Pmel−Trx and rTrx treated Pmel T cells exhibit increased pAMPK compared to activated Pmel T cells, and supports that maintaining high Trx levels on T cells is essential for “metabolic fitness” of T cells. Importantly, qPCR analysis for ‘stemness’ genes also showed up-regulation of the Lef1 and Tcf7 genes in Pmel T cells that were activated in presence of recombinant Trx (FIG. 7G). Additionally, compared to cognate antigen activated Pmel T cells (light blue overlay), Pmel T cells cultured with recombinant Trx for three days (orange overlay) exhibit reduced cell death as measured by Annexin V levels (FIG. 7H), and glucose uptake (similar to Pmel−Trx T cells in dark blue overlay) (FIG. 7I) upon TCR re-stimulation with cognate antigen (hgp100). Thus, this data indicates that restoring high Trx levels on immediate effector T cells could be important to render the memory phenotype with unique differentiation program (as high AMPK, low glucose uptake) (Blagih J et al., 2015, Immunity, 42(1):41-54; D'Souza W N et al., 2006, J Immunol, 177(2):777-81).

Further, it was determined if the strategy to activate and expand in presence of rTrx ex vivo would render tumor epitope reactive T cells with robust anti-tumor property. The data shows that gp100 reactive effector T cells generated in presence of rTrx do survive longer in vivo after adoptive transfer in a C57BL/6 host bearing subcutaneous B16-F10 murine melanoma and lead to much improved tumor control (FIG. 7J). This data shows that rTrx cultured T cells could keep their functional phenotype in vivo, and similar strategies could be employed in clinical scenario where TCR transduced T cells or chimeric antigen receptor (CAR) transduced patient T cells could be reprogrammed to improve their anti-tumor function.

### Human T Cells Engineered to Express Trx Exhibit Enhanced Anti-Oxidant Levels and Central Memory Phenotype

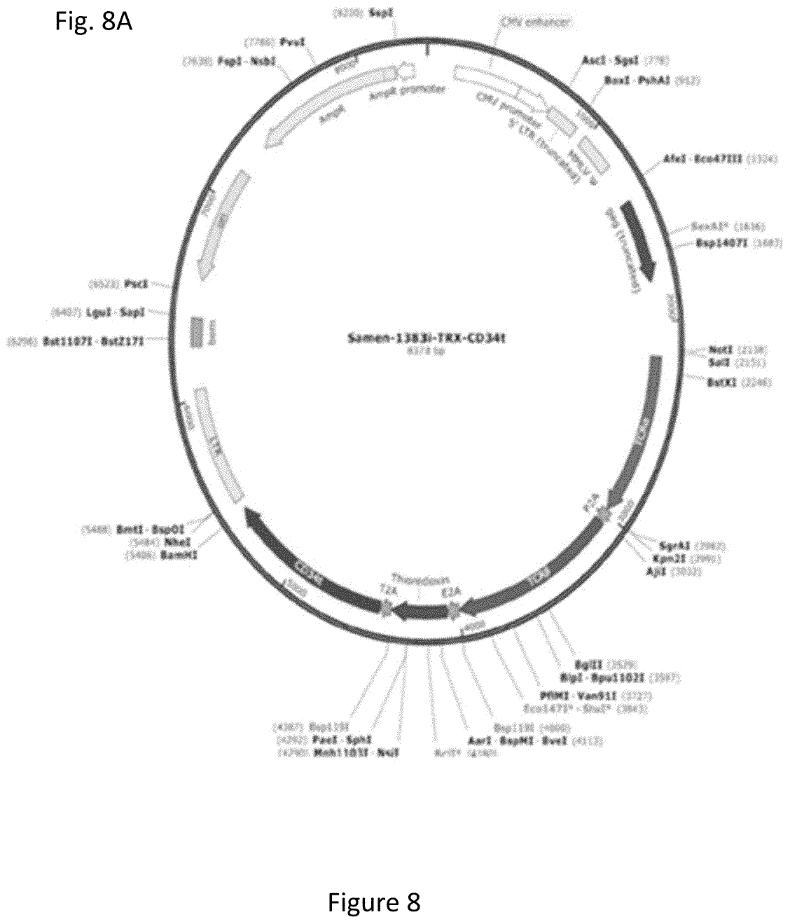

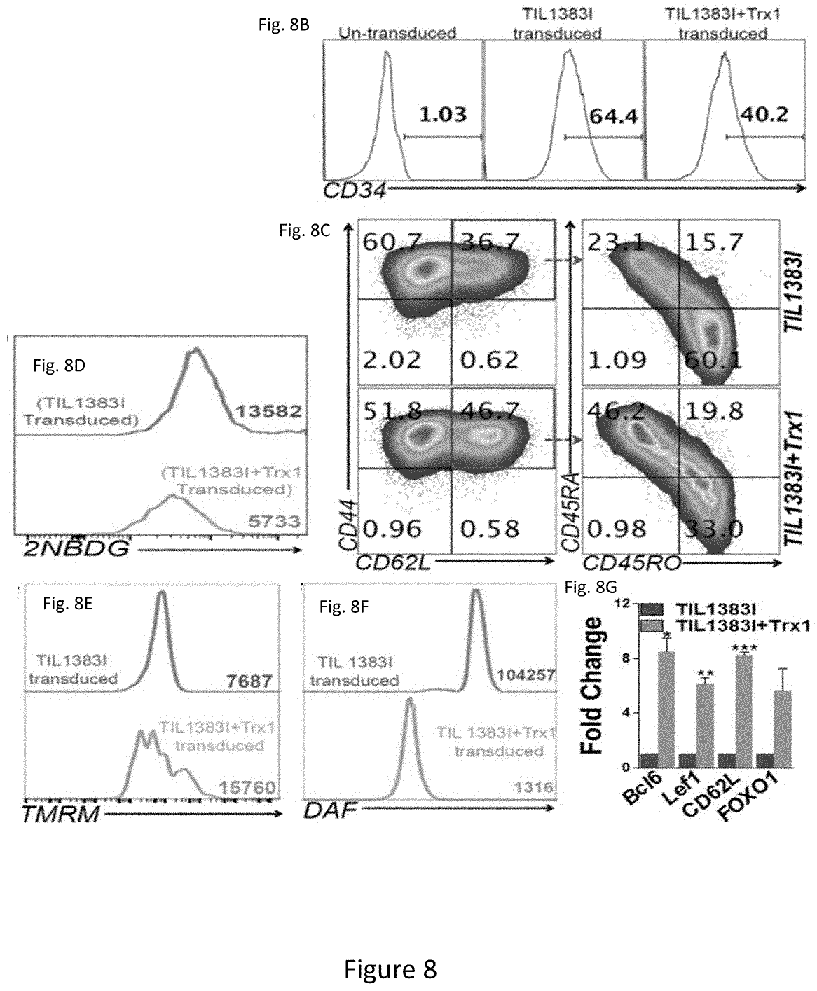

T cells from human patients are being used for adoptive immunotherapy approaches after engineering them with tumor reactive T cell receptors (TCR) or chimeric antigen receptors (CARs) (Rosenberg S A et al., 2015, Science, 348(6230):62-8). In order to determine if the strategy to increase anti-oxidant property of T cells will render human T cells with altered phenotype (as observed in mouse studies) a human melanoma epitope tyrosinase reactive TIL1383I retroviral construct with human Trx inserted to it was generated. For this purpose the gene construct was synthesized (at Genscript) with a Trx gene flanked by Bsp119i restriction sites and then cloned into the original Samen/1383I-34t vector. The clones were screened for correct orientation (FIG. 8A). The retroviral supernatant was used to transduce the activated human T cells with either TIL1383I TCR or TIL1383I-TCR+Trx. FIG. 8B shows that retroviral construct with Trx could be used to generate tyrosinase epitope reactive T cells with transduction efficiency of 40% or more. Majority of the expanded cells exhibited CD62L+CD44+CD45RA+ Tscm phenotype (FIG. 8C) (Gattinoni L et al., 2011, Nat Med, 17(10):1290-7; Flynn J K et al., 2014, Clin Transl Immunology, 3(7):e20; Graef P et al., 2014, Immunity, 41(1):116-26). Importantly, engineering Trx on human T cells also resulted in reducing the glycolytic commitment as observed by lower glucose uptake in 2NBDG assay (FIG. 8D). Further, upon overnight TCR re-stimulation with cognate antigen the TIL1383I-Trx transduced T cells showed less cell death as indicated by higher mitochondrial membrane potential (FIG. 8E), that also co-related with reduced NO accumulation (FIG. 8F). A qPCR based analysis also showed that TIL1383I-Trx transduced T cells express significantly higher level of ‘stemness’ genes as compared to the TIL1383I TCR transduced T cells (FIG. 8G, p<0.005). This data establishes that the antitumor effector T cells can be programmed ex vivo for increasing anti-oxidant phenotype that could translate to better tumor control in vivo.

### SUMMARY

It has long been known that lymphocytes require a reducing milieu for optimal activation/proliferation (Angelini G et al., 2002, Proc Natl Acad Sci USA, 99(3):1491-6). It has been shown that T lymphocytes are defective in cysteine uptake and thus require exogenous thiols for activation and function (Angelini G et al., 2002, Proc Natl Acad Sci USA, 99(3):1491-6). As the functional group of the amino acid cysteine, the thiol (—SH) group plays a very important role in biology (Haugaard N, 2000, Ann N Y Acad Sci, 899:148-58). Recently, oxidative cysteine modifications have emerged as a central mechanism for dynamic post-translational regulation of almost all major protein classes, and correlate with many disease states (Leonard S E et al., 2011, Curr Opin Chem Biol, 15(1):88-102). Certain proteins in which the redox state of cysteine residues are modified (termed ‘redox sensors’), seem to be involved in the initial and direct regulation of signaling molecules in response to ROS (Ray P D et al., 2012, Cell Signal, 24(5):981-90). Such ‘redox sensors’ commonly possess highly conserved free cysteine (Cys) residues of which the —SH functional groups are the most important direct cellular targets or ‘sensors’ of ROS (Janssen-Heininger Y M et al., 2008, Free Radic Biol Med, 45(1):1-17). A number of ‘redox sensors’ have been identified that participate in many important biological functions, some of which are crucial molecules modulating stem cell self-renewal and differentiation, including HIF-1α, FoxOs, APE1/Ref-1, Nrf2, AMPK, p38 and p5351-54. In addition, glutamate (Glu) and anti-TRX-inactivating antibodies inhibit antigen-dependent T lymphocyte proliferation (Angelini G et al., 2002, Proc Natl Acad Sci USA, 99(3):1491-6). In T lymphocytes, intracellular GSH is critical for the proliferative response to mitogens or antigens (Messina J P et al., 1989, J Immunol, 143(6):1974-81; Suthanthiran M et al., 1990, Proc Natl Acad Sci USA, 87(9):3343-7; Mihm S et al., 1995, FASEB J, 9(2):246-52; Smyth M J, 1991, J Immunol, 146(6):1921-7). However, lymphocytes lack an efficient system of Cys2 import, whereas they easily take up free thiols (Ishii T et al., 1987, J Cell Physiol, 133(2):330-6; Gmunder H et al., 1990, Cell Immunol, 129(1):32-46). Therefore, to sustain lymphocyte activation and proliferation, exogenous thiols must somehow be generated in the microenvironment of an immune response. Extracellular thioredoxin (Trx) has been proposed to exert a synergistic activity on the mitogen- or cytokine-induced proliferation of lymphocytes (Wakasugi N et al., 1990, Proc Natl Acad Sci USA, 87(21):8282-6; Iwata S et al., 1994, J Immunol, 152(12):5633-42). It is shown herein that increasing the Trx in tumor microenvironment by co-injecting Trx over-expressing T cells or using rTrx during ex vivo programming maintains the reducing environment and leads to long-term T cell anti-tumor function in vivo.

The present data indicates the functional differences between the CD8+ T cells obtained from the Pmel and Pmel−Trx mice, likely due to the protein thiol alterations that remains unknown at this time. Oxidation of thiol (—SH) groups is a post-translational modification that regulates numerous processes, including differentiation, cellular proliferation and apoptosis (Davis W, Jr., et al., 2001, J Pharmacol Exp Ther, 296(1):1-6). Without wishing to be bound by any particular theory, it is hypothesized that the number of free reduced vs. oxidized thiols present on signaling molecules could lead to differences in their functionality, and thus dictate the effector T cells vs. memory T cell phenotype. The data shows that the difference observed between the Pmel and Pml-Trx T cells is exclusively due to Trx, since addition of recombinant Trx led to a decrease in NF-KB activity. While increased activation of NF-KB has been shown to be important for the robust effector function of T cells (Ruan Q et al., 2012, Adv Exp Med Biol, 946:207-21), it has also been shown that persistent activation of NF-KB could lead to enhanced replicative senescence and cell death (Vaughan S et al., 2011, Aging (Albany N.Y.), 3(10):913-9).

The commitment to different metabolic pathways could lead to differences in level of intrinsic metabolites in a cell, which could be important in regulating various signaling pathways (Metallo C M et al., 2010, Genes Dev, 24(24):2717-22). The data quantifying the metabolite levels between TCR activated Pmel vs. Pmel−Trx T cells show distinct profile in thioredoxin over expressing T cells. The activated Pmel−Trx cells exhibit increased level of pentose phosphate pathway (PPP) metabolites that contribute to nucleotide precursors and helps regenerate the reducing agent NADPH, which can contribute to ROS scavenging. In addition, the tricarboxylic acid cycle (TCA, also known as Krebs's cycle) metabolite alpha-ketoglutarate (α-KG) was also found to be significantly elevated in the Pmel−Trx cells. The role of α-KG, also produced by deamination of glutamate, in the detoxification of ROS has only recently begun to be appreciated (Lemire J et al., 2010, FEMS Microbiol Lett, 309(2):170-7). This keto-acid neutralizes ROS in an NADPH-independent manner with the concomitant formation of the succinate and CO₂. In addition, α-KG has also been shown to extend the lifespan of adult C. elegans (Chin R M et al., 2014, Nature, 510(7505):397-401). This study showed that α-KG inhibits ATP synthase leading to reduced ATP content, decreased oxygen consumption, and is dependent on the target of rapamycin (TOR) downstream. Further, the role of metabolite α-KG has also been shown in maintaining the pluripotency of the embryonic stem cells (Carey B W et al., 2015, Nature, 518(7539):413-6). Thus, it is likely that Trx mediated differences in metabolic pathways that lead to difference in accumulation of metabolites (such as α-KG) may have led to epigenetic reprogramming of T cells resulting in sustained tumor control and memory generation.

The observation that Trx overexpressing T cells exhibit increased glutamine uptake also implies that this amino acid may have contributed towards the programming of Pmel−Trx effectors for enhanced anti-tumor phenotype. It has also been shown that while glutamine helps differentiation of T cells to effector phenotype (34), and the deficiency of glutamine can result in formation of Treg (Klysz D et al., 2015, Sci Signal, 8(396):ra97). Importantly, higher glutamine levels in Pmel−Trx T cells would have led to down-regulation of CD95 and CD95L expression, and up-regulation of memory marker CD45RO and Bcl-2 expression as has been shown earlier (Chang W K et al., 2002, Clin Immunol, 104(2):151-60). Thus, the results presented here in support the role of Trx in regulating redox status of adoptively transferred T cells, and that Trx mediated “anti-oxidant help” in vivo can be important in generating long-lived anti-tumor memory T cells in the oxidative tumor microenvironment in vivo. The strategy to generate ‘anti-tumor memory T cells’ using Trx will have great translational significance in the field of cancer immunotherapy.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1-5. (canceled)

6. A method of treating cancer in a mammal, the method comprising administering an effective amount of a reprogrammed T cell to a mammal in need thereof, wherein the reprogrammed T cell exhibits a higher expression of cell surface thiols, wherein the T cell is reprogrammed by culturing the cell with one or more pharmacological modulators.

7. A method for stimulating an immune response to a target cell population or tissue in a mammal, comprising administering to a mammal an effective amount of a reprogrammed T cell that exhibits higher expression of cell surface thiols, thereby stimulating a response to a target cell population or tissue in the mammal, wherein the T cell is reprogrammed by culturing the cell with one or more pharmacological modulators.

8. A method of providing an anti-tumor immunity in a mammal, the method comprising administering to the mammal an effective amount of a reprogrammed T cell that exhibits expression of cell surface thiols, thereby providing an anti-tumor immunity in the mammal, wherein the T cell is reprogrammed by culturing the cell with one or more pharmacological modulators.

9. (canceled)

10. The method of claim 6, wherein the cancer is selected from the group consisting of melanoma, lung cancer, ovarian cancer, head and neck cancer, breast cancer, prostate cancer, cervical cancer, skin cancer, pancreatic cancer, colorectal cancer, renal cancer, liver cancer, brain cancer, lymphoma, leukemia, and lung cancer.

11. The method of claim 6, wherein the reprogrammed T cell persists in vivo for at least four months, five months, six months, seven months, eight months, nine months, ten months, eleven months, twelve months, two years, or three years after administration.

12. The method of claim 6, wherein the method further comprises administration of one or more selected from the group consisting of a second anti-tumor agent, an antiproliferative agent, and a chemotherapeutic agent.

13. The method of claim 6, wherein the one or more pharmacological modulator is selected from the group consisting of IL-4, recombinant thioredoxin (rTrx), thioredoxin-reductase, glutathione, α-ketoglutarate, and a combination thereof.

14. The method of claim 13, wherein the one or more pharmacological modulator is recombinant thioredoxin (rTrx).

15. The method of claim 7, wherein the one or more pharmacological modulator is selected from the group consisting of IL-4, recombinant thioredoxin (rTrx), thioredoxin-reductase, glutathione, α-ketoglutarate, and a combination thereof.

16. The method of claim 15, wherein the one or more pharmacological modulator is recombinant thioredoxin (rTrx).

17. The method of claim 8, wherein the one or more pharmacological modulator is selected from the group consisting of IL-4, recombinant thioredoxin (rTrx), thioredoxin-reductase, glutathione, α-ketoglutarate, and a combination thereof.

18. The method of claim 17, wherein the one or more pharmacological modulator is recombinant thioredoxin (rTrx).
